(19) United States
(12) Patent Application Publication
(10) Pub. No.: US 2010/0136645 A1
(43) Pub. Date: Jun. 3, 2010

(54) METHOD FOR PREPARING A CELL-DERIVED EXTRACELLULAR MATRIX SCAFFOLD

(75) Inventors: Byoung-Hyun Min, Seoul (KR); So Ra Park, Seoul (KR); Cheng Zhe Jin, Gyeonggi-do (KR); Kwideok Park, Gyeonggi-do (KR)

Correspondence Address: LUCAS & MERCANTI, LLP, 475 PARK AVENUE SOUTH, 15TH FLOOR, NEW YORK, NY, 10016, US

(73) Assignee: Byoung-Hyun MIN, Seoul (KR)

(21) Appl. No.: 12/596,008

(22) PCT Filed: Apr. 17, 2007
(86) PCT No.: PCT/KR2007/001873

§ 371 Date: Dec. 14, 2009

(51) Int. Cl. C12N 13/00; C12N 5/02

(52) U.S. Cl. 435/173.1; 435/401; 435/180

(57) ABSTRACT

The present invention relates to a method for fabricating a cell-derived extracellular matrix scaffold, more particularly, to a method for fabricating a cell-derived extracellular matrix scaffold, the method comprising the steps of obtaining a chondrocyte/extracellular matrix (ECM) membrane from chondrocytes derived from animal cartilage, obtaining a pellet-type scaffold-free construct by culturing after centrifuging the obtained chondrocytes/extracellular matrix (ECM) membrane and freeze-drying the obtained pellet-type construct. The cell-derived ECM scaffold according to the invention is a porous scaffold fabricated using cartilage tissue engineered by culturing chondrocytes in an in vitro scaffold-free system, which is not reduced in size during the cultivation and thus useful for cartilage regeneration.

### TECHNICAL FIELD

The present invention relates to a method for fabricating a cell-derived extracellular matrix scaffold, more particularly, to a method for fabricating a cell-derived extracellular matrix scaffold, the method comprising the steps of obtaining a chondrocyte/extracellular matrix (ECM) membrane from chondrocytes derived from animal cartilage, obtaining a pellet-type scaffold-free construct by culturing after centrifuging the obtained chondrocytes/extracellular matrix (ECM) membrane, and freeze-drying the obtained pellet-type construct.

### BACKGROUND ART

Articular chondrocytes are specialized mesoderm-derived cells found in only cartilage. Cartilage is an avascular tissue having physical properties depending on the properties of ECM produced by chondrocytes. During cartilage generation, chondrocytes become mature to cause the initiation of chondrocyte hypertrophy coinciding with the onset of type X collagen expression (Stephens, M. et al., J. Cell Sci., 103:1111, 1993).

Autologous chondrocyte implantation (ACI) used for treating cartilage defects is a clinically approved cell transplantation to regenerate normal hyaline cartilage in the area of the cartilage defect (Brittberg, M. et al., New Eng. J. Med., 331:889, 1994). Cell transplantation using a variety of scaffolds and advanced methods for fabricating tissue engineering cartilage in vitro have been developed, with the advancement of studies on chondrocytes and mesenchymal stem cells (MSCs) (Lee, C. R. et al., Tissue Eng., 6:555, 2000, Li, W. J. et al., Biomaterials, 26:599, 2005).

Scaffolds which provide a three-dimensional (3D) culture environment affect not only proliferation and differentiation of seeded cells but also the ultimate quality of tissue-engineered cartilage tissues. At present, various substances synthesized or derived from natural materials are used as appropriate scaffolds. These scaffolds have been utilized in various forms, such as sponges, gels, fibers, microbeads and so forth (Honda, M. J. et al., J. Oral Maxillofac Surg., 62:1510, 2004, Griogolo, B. et al., Biomaterials, 22:2417, 2001, Chen, G. et al., J. Biomed. Mater. Res. A, 67:1170, 2003, Kang, S. W. et al., Tissue Eng., 11:438, 2005). Among them, the most commonly used one is a porous structure which is capable of enhancing cell adhesion activity and has high surface tension to maintain volume. Although some successful applications thereof in vivo and in vitro have been reported, it was difficult to produce a-high quality tissue-engineered cartilage, so that there is a problem to apply them in clinical practice. Therefore, it is needed to improve scaffolds in structural and functional aspects so as to solve the problem.

Accordingly, the present inventors considered that successful treatment for regenerating hyaline cartilage tissue can be achieved if an ECM membrane, which is a structurally complicated but well-organized compound of various natural proteins in a three-dimensional structure, is used as a scaffold.

Previously, allogenic or xenogenic ECM membrane was directly harvested from a living tissue and acellularized to use as membrane-type scaffolds. Representative examples are small intestine submucosa (SIS), urinary bladder submucosa (UBS), human amniotic membrane (HAM) and the like. HAM is useful for cornea regeneration, and SIS is used for the regeneration of urinary tract and dura mater, and vascular reconstruction. And, studies on cartilage regeneration using type I, III collagen bilayer membrane, are also being conducted.

A chondrocyte-derived ECM scaffold consists basically of glycosaminoglycan (GAG) and collagen, which are main components of the extracellular matrix of cartilage tissue, and includes microelements which are important in chondrocyte metabolism. ECM scaffold provides a natural environment for chondrocyte differentiation and can be applied to the tissue-engineering field as a high quality scaffold.

Recently, a number of patents have been published that describe the followings; injectable chondrocyte implant (KR10-2004-7017580), porous scaffolds for tissue engineering comprising biodegradable Glycolide/ε-Caprolactone copolymer (KR10-0408458B), a method for producing a neutralized chitosan sponge for wound dressing and tissue-engineered scaffolds, and a neutralized chitosan sponge produced by the same (KR10-2003-0023929) and naturally secreted ECM composition and a method for using thereof (KR10-1997-708695), but they have problems in that, the preparation process thereof is complicated since scaffolds derived from a living tissue should undergo decellularization in a detergent solution, have low cell adhesion efficiency due to too high hardness and low porosity thereof, and generate an unsuitable transplant tissue that doesn't fit the defect due to the contraction of cell-seeded scaffolds and in vivo transplant tissue, or may even be separated from host tissue due to the looseness of the transplant tissue.

To solve the above mentioned problems, the present inventors have made extensive efforts to develop an ECM scaffold which can be fabricated in vitro, has proper hardness, high porosity and no abnormal response when transplanted into tissue, and can be applied to clinical use without causing contraction of cartilage tissue after transplantation, and as a result, fabricated a porous ECM scaffold using a method in which a tissue-engineered cartilage is prepared using chondrocytes in vitro and the chondrocytes were removed to freeze-dry the tissue-engineered cartilage, and confirmed that the ECM scaffold did not cause tissue contraction after transplantation and can maintain cell differentiation for a long time, thereby completing the present invention.

### SUMMARY OF THE INVENTION

Accordingly, an object of the present invention is to provide a method for tissue-engineered fabrication of an ECM scaffold in an in vitro scaffold-free system.

Another object of the present invention is to provide a porous ECM scaffold which can maintain cell differentiation for a long time, and be applied in the fields of clinical practice and cartilage tissue-engineering.

In order to achieve the above objects, the present invention provides a method for fabricating a cell-derived ECM scaffold, the method comprising the steps of: (a) isolating chondrocytes from animal cartilage and then culturing them; (b) obtaining a chondrocyte/ECM membrane from the cultured chondrocytes; (c) obtaining a pellet-type scaffold-free construct by culturing the obtained chondrocyte/ECM membrane; and (d) obtaining an ECM scaffold by freeze-drying the obtained pellet-type construct.

The present invention also provides a method for fabricating a cell-derived ECM scaffold, the method comprising the steps of: (a) isolating chondrocytes from animal cartilage and then culturing them; (b) obtaining a chondrocyte/ECM membrane from the cultured chondrocytes; and (c) obtaining an ECM scaffold by folding the obtained chondrocyte/ECM membrane or by overlapping several membranes.

The present invention also provides a cell-derived porous ECM scaffold fabricated by the method, which is not shrunken in size during tissue culture and has pores with a diameter of 10˜1000 μm.

The present invention also provides a method for fabricating an ECM scaffold similar to natural cartilage or having an excellent mechanical intensity, in which cartilage components are added to the ECM scaffold and mixing them.

The present invention also provides a method for fabricating an ECM composite scaffold in which biodegradable polymers are attached to the ECM scaffold.

Another features and embodiments of the present invention will be more clarified from the following detailed description and the appended claims.

### BRIEF DESCRIPTION OF DRAWINGS

FIG. 1 shows the final morphology of extracellular matrix scaffolds according to the present invention, and scale bar represents one unit of one mm.

FIG. 2 is SEM (scanning electron microscope) images of a periphery region (A) and a core (B) region of the ECM scaffold according to the present invention (original magnification, 30×), and the arrow represents a highly compact region.

FIG. 3 shows the effect of the initial cell-seeding density on both the number and adhesion rate of cells attached to the ECM scaffold according to the present invention, and *represents statistical significance.

FIG. 4 is SEM images of chondrocyte morphology at 0 hour (A, C) and 12 hours (B, D) post-seeding into the inventive ECM scaffold (original magnification, 200× and 1000×, respectively). Herein, the white arrow shows morphological changes of cells depending on culture time.

FIG. 5 is images of neocartilage formed in the inventive ECM scaffold seeded with chondrocytes cultured in vitro, which are observed by naked eyes. Scale bar represents one unit of one mm and W represents a week.

FIG. 6 is images showing the results of histological assessment of cartilage tissue engineered by culturing chondrocytes in vitro for 1, 2 and 4 weeks (original magnification, 20× and 200×, respectively). Herein, the black arrow shows changes in scaffold wall thickness depending on culture time.

FIG. 7 is images showing the results of immunohistochemical assessment of cartilage tissue engineered by culturing chondrocytes in vitro for 1, 2 and 4 weeks (original magnification, 20× and 200×, respectively). G is a negative control untreated with primary antibody and H is a positive control treated with primary and secondary antibodies (original magnification, 200×).

FIG. 8 is western blot images of type I, II collagen by electrophoresis.

### DETAILED DESCRIPTION OF THE INVENTION, AND PREFERRED EMBODIMENTS

In one aspect, the present invention relates to a method for tissue-engineered fabrication of a cell-derived ECM scaffold in an in vitro scaffold-free system.

A first embodiment of the method for fabricating the ECM scaffold according to the present invention includes the steps of: (a) isolating chondrocytes from animal cartilage and then culturing them; (b) obtaining a chondrocyte/ECM membrane from the cultured chondrocytes; (c) obtaining a pellet-type scaffold-free construct by culturing the obtained chondrocyte/ECM membrane; and (d) obtaining an ECM scaffold by freeze-drying the obtained pellet-type construct.

A second embodiment of the method for fabricating the ECM scaffold according to the present invention includes the steps of: (a) isolating chondrocytes from animal cartilage and then culturing them; (b) obtaining a chondrocyte/ECM membrane from the cultured chondrocytes; and (c) obtaining an ECM scaffold by folding the obtained chondrocyte/ECM membrane or by overlapping several membranes.

In the present invention, the animal is preferably a pig, and the culture step preferably, additionally comprises adding growth factors so as to enhance the strength of an ECM scaffold as well as make components and structure of the scaffold similar to natural cartilage. The growth factors are preferably selected from the group consisting of IGF (insulin-like growth factor), FGF (fibroblast growth factor), TGF (transforming growth factor), BMP (bone morphogenetic protein), NGF (nerve growth factor) and TNF-α (tumor necrosis factor-alpha), but not limited thereto. Also, in the culture step, preferably, culture broth is treated with ultrasonic waves or physical pressure is applied to the culture broth so as to facilitate chondrocyte proliferation and ECM secretion.

In the present invention, in order to increase the size of a well-fabricated ECM scaffold, the ECM scaffold may be fabricated by obtaining chondrocyte/ECM membranes from cells cultured in more than two test tubes, mixing them to centrifuge, and culturing them in a large culture plate (e.g., 150 mm culture plate). Such fabricated large-sized ECM scaffold has much higher possibility of being applied in clinical practice.

In the first embodiment of the present invention, the step (c) is preferably performed by fractionating the chondrocyte/ECM membrane to collect and culturing them, the step (d) is preferably performed by repeating 3˜5 times a cycle of freezing and thawing the pellet-type construct at −15˜−25° C. to freeze-dry, and the method preferably includes an additional step (e) of obtaining a disk-shaped ECM scaffold by processing the obtained ECM scaffold.

In another aspect, the present invention relates to a cell-derived porous ECM scaffold fabricated by the method, which is not reduced in size during tissue culture and has pores with a diameter of 10˜1000 μm, and its application.

For example, when cartilage components are added to the ECM scaffold to mix them, an ECM scaffold, which is similar to natural cartilage or has an excellent mechanical intensity, can be fabricated. Therefore, in still another aspect, the present invention relates to a method for fabricating an ECM scaffold similar to natural cartilage or having an excellent mechanical intensity, in which cartilage component is added to the ECM scaffold to mix them as well as a method for fabricating an ECM composite scaffold in which biodegradable polymers are attached to the ECM scaffold.

In the present invention, the cartilage component is preferably collagen or proteoglycan, but not limited thereto.

Moreover, when biodegradable polymers are attached to the ECM scaffold, an ECM composite scaffold can be fabricated for cartilage regeneration as well as bone regeneration or bone/cartilage regeneration. In the present invention, the biodegradable polymers can be preferably selected from the group consisting of collagen, PLGA (poly-lactic-co-glycolic acid), PLA (polylactate) and PHA (polyhydroxyalkanoate), but not limited thereto.

In the present invention, a cell-derived ECM scaffold composed of chondrocytes and their self-produced ECM, which is capable of providing an optimal 3D environment where chondrocytes can grow and develop into a high quality cartilage tissue, was fabricated.

In order to fabricate the ECM scaffold according to the present invention, chondrocytes isolated from pig cartilage were monolayer-cultured at a high density for 3-4 days, and then pellet-type scaffold-free cartilage constructs were obtained by centrifuging the obtained chondrocyte membrane, followed by culturing them in vitro for 3 weeks. After the cultured constructs were freeze-dried, new ECM scaffolds were fabricated by processing them at maximum rate with a biopsy punch in order to make the cartilage-specific ECM-containing scaffold in the form of disks.

As a result of observing the surface structure of the fabricated ECM scaffold using SEM, the ECM scaffold according to the present invention had a lower density than that of natural cartilage matrix, and showed the results of an average (n=6) porosity of 90±10.4%, an average (n=6) pore size of 113±26 μm, a porosity rate of 89.1±8.3% and a compressive strength of 0.34±0.09 MPa.

Western blot analysis (by SDS-PAGE) revealed that the collagen type expressed in the ECM scaffold according to the present invention was type II collagen. Also, the amount of GAG and collagen in the ECM scaffold according to the present invention was measured and compared to those in natural cartilage tissue. The total GAG content was 108.1±19.1 μg/mg and the collagen content was 53.8±6.7 μg/mg (dry weight), corresponding to ⅓ of the natural cartilage tissue.

Rabbit chondrocytes (P1) whose phenotypes were maintained were dynamically seeded into the ECM scaffold according to the present invention for histological analyses using SEM. The result exhibited that the seeded chondrocytes were well attached to the scaffold wall and the cell adhesion rate was 58±6%. The ECM scaffold into which chondrocytes were seeded was cultured in vitro for 4 weeks, and the forming of cartilage tissue had been observed, which included morphology, volume, histology of the formed tissue at each time point of 1, 2, and 4 weeks, respectively. As a result, with the passage of time, it was detected that the surface of the cartilage-like tissue became gradually smooth and white in color as well as the intensity increased, whereas the volume was unchanged so that it was found that the significant contraction of the initial tissue size did not occur.

Also, the results from histological analyses using Safranin O and Alcian blue staining revealed that the sulfated proteoglycan (GAG) was accumulated continuously so that the inner pore spaces of the scaffold were fully charged. Type II collagen formed in the pericellular and pore regions was detected from immunohistochemical analysis. After extracting the total proteins from the neocartilage tissue, immunoblotting was performed, and as a result, it was found that a main ECM component was type II collagen in the tissue. This outcome exhibited that phenotypes of chondrocytes can be maintained and accumulated for a long time, and consequently supported that cell differentiation can be maintained for a long time in the ECM scaffold environment according to the present invention.

From the results, it was confirmed that the new ECM scaffold according to the present invention could provide a natural 3D environment to form excellent cartilage tissue in vitro and can be applied in the fields of clinical practice and cartilage tissue engineering.

### EXAMPLES

Hereinafter, the present invention will be described in more detail by examples. It will be obvious to a person skilled in the art, however, that these examples are for illustrative purpose only and are not construed to limit the scope of the present invention.

Particularly, the following examples describe a method for fabricating the ECM scaffold using pig articular cartilages according to the method of the present invention, however, it will be obvious to a person skilled in the art that an ECM scaffold is fabricated using cartilages of other animals.

Also, the following examples exemplify a method for fabricating the ECM scaffold according to the first embodiment of the present invention, however, it will be obvious to a person skilled in the art that an ECM scaffold is fabricated by folding the chondrocyte/ECM membrane or overlapping the membranes, obtained by the first embodiment. The folding means a process of making a given shape by folding chondrocyte/ECM membrane. Folding or overlapping allows to fabricate a more stereo structural scaffold from the pellet-type chondrocyte/ECM membrane.

Also, although the following examples do not contain concrete illustrations, it will be obvious to a person skilled in the art that an ECM scaffold similar to natural cartilage or having an excellent mechanical intensity can be fabricated by adding cartilage components such as collagen, proteoglycan to the inventive ECM scaffold and mixing them. Moreover, it will be obvious to a person skilled in the art that an ECM composite scaffold can be fabricated by attaching collagen or biodegradable polymers to the inventive ECM scaffold.

### Example 1

### Isolation of Chondrocytes

Articular cartilages were harvested from the stifles of 2- to 3-week-old pigs. The cartilage pieces were separated carefully from the other tissues and washed with phosphate-buffered saline (PBS), followed by treating them with 0.05% (wt/vol) Pronase (Boehringer, Mannheim, Germany) at 37° C. for 1.5 hours. They were washed twice with PBS and then subjected to treatment of 0.2% (wt/vol) collagenase (Worthington Biochemical Corp., Lakewood, N.J., USA) for 12 hours in Dulbecco's modified Eagle medium (DMEM) (Gibco, Grand Island, N.Y., USA) supplemented with 5% newborn calf serum (NCS) (Hyclone, Logan, Utah, USA). After the cartilage tissues were completely digested, the isolated chondrocytes were centrifuged at 600×g for 10 min. The precipitated chondrocytes were washed twice and seeded in tissue culture plates (100 mm diameter ×20 mm height) at a density of 1.9×10⁵ cells per plate.

### Example 2

### Preparation of Cartilage Tissue Constructs and In Vitro Culture

The chondrocytes isolated in Example 1 were cultured in monolayer using DMEM supplemented with 10% NCS (new-born calf serum), 50 units/ml penicillin 50 μg/ml streptomycin, and 50 μg/ml L-ascorbic acid for 3-4 days. After cultivation, the medium was removed and 0.05% Trypsin-ethylenediaminetetra acetic acid (Trypsin-EDTA) (Gibco) was added to obtain a chondrocyte/ECM membrane from the culture plate. The obtained membranes were isolated carefully with a wide-bore pipette and transferred individually to a 50 ml conical tube filled with 30 ml DMEM supplemented with 5% NCS. In order to make a pellet-type construct, each tube was then centrifuged at 600×g for 20 minutes and then incubated at 37° C. for 12 hours. The cultured constructs were transferred to a 6-well culture plate for a secondary culture for 3 weeks. From the cultivation process, 5 ml of the culture medium was replaced three times a week. As a result, the constructs grew into neocartilage tissue.

### Example 3

### Preparation of an ECM Scaffold

Neocartilage tissue constructs obtained in Example 2 through 3-week cultivation were washed with PBS and then stored at −20° C. for 3 days. After repeating the process of freeze and thaw three times, the constructs were freeze-dried for 48 h at −56° C. under 5 m Torr. Using a biopsy punch (6 mm diameter), the freeze-dried specimens were split into two parts, which are a disk-shaped core and a ring-shaped periphery. Due to the dimensional consistency of the core region, the disk-shape was chosen as a preform of the ECM scaffold. By additional process, the preformed substance was further trimmed off the surface layer by less than 1 mm in thickness, thus resulting in the final form of the ECM scaffold (FIG. 1).

If 3-week cultured neocartilage constructs are freeze-dried, they are transformed into a sponge type with suitable hardness because the core region of freeze-dried specimens was separated from the periphery region using a 6 mm biopsy punch, not due to the irregular shape of freeze-dried specimens (˜8 mm diameter). Accordingly, a disk-shaped preform of an ECM scaffold was prepared (FIG. 1A).

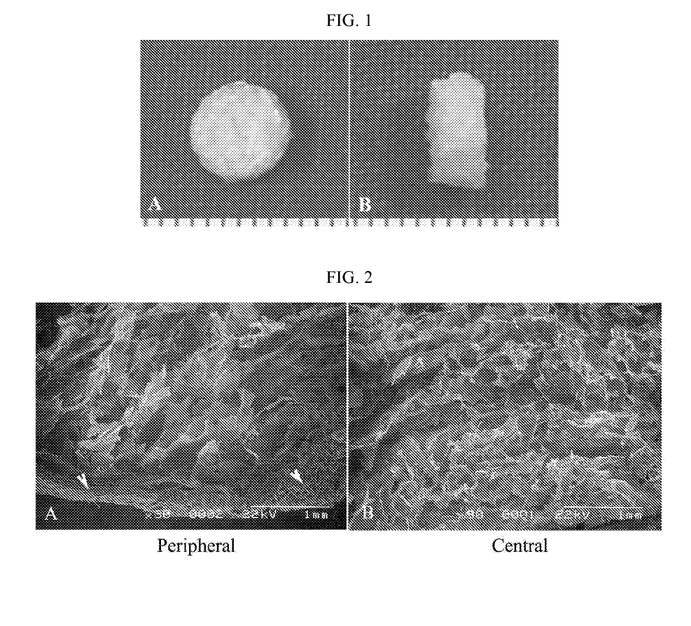

FIG. 2 is SEM images of the periphery (A) and core (B) regions in an ECM scaffold, and it was revealed that the peripheral layer of freeze-dried cartilage constructs has the unsuitable shape without porosity for cell seeding. Because the peripheral layer of the preform scaffold analyzed by SEM, as shown as an arrow in FIG. 2A, is highly compacted, the seeded chondrocytes could not pass through the inner region. Therefore, in order to fabricate the porous ECM scaffold, the peripheral layer was required to be trimmed off to expose a highly porous microstructure over the whole region (FIG. 2B).

### Example 4

### Biochemical Analysis of Total Glycosaminoglycan (GAG) and Collagen Contents

In order to measure the GAG and collagen contents of the ECM scaffold fabricated in Example 3, the ECM scaffold was digested in papain solution (5-mM L-cysteine, 100 mM Na₂HPO₄, 5 mM EDTA, 125 μg/ml papain type III, pH 7.5) at 60° C. for 24 hours and then centrifuged at 12,000×g for 10 min.

In order to measure the GAG contents of the supernatant, dimethylmethylene blue (DMB) colorimetric assay (Heide, T. R. and Gernot, J., Histochem. Cell Biol., 112:271, 1999) was performed, and the total collagen contents were measured using Heide tullberg-reinert method (Schmidt, C. E. and Baier, J. M., Biomaterials, 22:2215, 2000).

After the digested specimens were dried at 37° C. in a 96-well plate, they were reacted with 1 mg/ml sirius red collagen-staining solution (pH3.5) dissolved in a picric acid saturation solution (1.3%, Sigma, Mo., USA) in a stirrer for 1 hour. The stain-specimen in each well was washed with 0.01N HCl five times and then dissolved in 0.1N NaOH, thereby measuring its absorbance at 550 nm wavelength using an ELISA READER (BIO-TEK, Instruments, Inc., USA).

As a result, the total GAG and collagen contents analyzed biochemically were 108.1±19.1 μg/mg (dry weight) and 53.8±6.7 μg/mg (n=6), respectively, reaching ⅓ of natural cartilage tissue.

### Example 5

### Measurement of Mechanical Characteristic

Mechanical compressive strength of the ECM scaffold fabricated in Example 3 was measured using a Universal Testing Machine (model H5K-T, H.T.E., England). Before the measurement, the specimens (n=6) were cut into uniform rectangular shapes and pulled at a crosshead speed of 1 mm/min with both ends of a specimen grasped. A peak load was obtained from the load-displacement curve at break and then individual compressive strengths were calculated. Non-woven mesh, PGA scaffold (Albany international, NY, USA) was used as a control group (Table 1).

TABLE 1
Mechanical characteristic

| | ECM scaffold | Mesh PGA scaffold | |
|---|---|---|---|
| | (n = 6) | (n = 6) | P value |
| Compressive strength | 0.34 ± 0.09 | 0.52 ± 0.07 | 0.018 |
| (Mpa) | | | |

Mechanical characteristic of the ECM scaffold according to the present invention was presented in Table 1. The maximum compressive strength measured by pulling specimens one-axially was averagely 0.34±0.09 MPa (n=6). Although the compressive strength of the ECM scaffold according to the present invention was lower than that of the commercialized PGA scaffold, it was found that the ECM scaffold was preserved without any defects or damages during the whole fabrication process because it had a sustained hardness. When using a method for obtaining the improved hardness of natural scaffolds by crosslinking (Pieper, J. S. et al., Biomaterials 21:581, 2000), compressive strength of the ECM scaffold according to the present invention can increase. Also, mechanical strength can be multiplied by adding cartilage components like collagens and proteoglycan to the ECM scaffold according to the present invention and mixing them.

### Example 6

### Determination of Proper Cell-Seeding Density and Cell Adhesion Rate

The ECM scaffolds fabricated in Example 3 were soaked in sterile 70% ethanol for 1 hour and washed with PBS, and then immersed in DMEM for 12 hours prior to the cell seeding. In order to determine the ideal seeding concentration, the rabbit chondrocytes (P1), whose phenotypes were maintained, were seeded dynamically on the ECM scaffolds (n=5) at 4 different densities of 1, 2, 3 and 4×10⁶ cells/ml for 1.5 hours with a nutator. The separated cells in a medium and a plate wall were totalized and attached cell number, and cell adhesion rate were examined at 1-hour post-seeding. After determining the cell density for seeding, the cells were seeded at a suitable density and the cell-seeded scaffolds were cultivated for 1, 2 and 4 weeks. As mentioned in Example 2, the same culture medium was used and replaced three times a week.

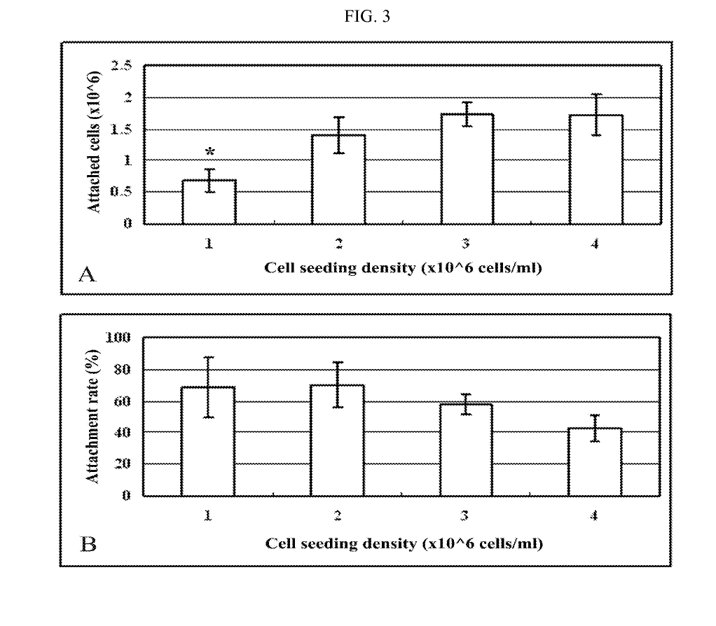

FIG. 3 shows the effect of the initial cell-seeding density on both the number and adhesion rate of cells attached to an ECM scaffold. After the rabbit chondrocytes (P1) whose phenotypes were maintained were seeded dynamically on the ECM scaffolds (n=5) at 4 different densities of 1, 2, 3 and 4×10⁶ cells/ml, the cell number attached within 1 hour was measured and it was found that along with the increase of the seeding density, the attached cell number increased to reach 0.7±0.2×10⁶, 1.4±0.3×10⁶, 1.7±0.2×10⁶ and 1.7±0.3×10⁶ cells/ml, respectively (FIG. 3A). There was no statistically significant difference between the measured control groups except the seeding concentration at the cell density of 1×10⁶ cells/ml.

Moreover, the cell adhesion rate was calculated on the basis of two factors, the detached cell number and the total seeded cell number. FIG. 3B exhibited that the average cell adhesion rate (%) was inversely proportional to the increase of the cell seeding density, presenting 69±19%, 70±14%, 58±6% and 43±8%, respectively.

From the results, it was confirmed that the cell adhesion rate was not concordant with the ideal range of cell number and the initial seeding density. That is, it is considered that there was no correlation between the cell seeding density and the cell adhesion rate. Therefore, it is estimated that as many cells as possible which were seeded on the scaffold might be advantageous. Although the average cell adhesion rate was not the highest, the cell seeding density of 3×10⁶ cells/ml was used in the present invention.

### Example 7

### Porosity and Pore Size of an Ecm Scaffold

The porosity and pore size of an ECM scaffold were measured using a mercury intrusion porosimeter (Micromeritics Co., Model AutoPore II 9220, USA). After the scaffold was placed in a chamber, the chamber was sealed tightly and vacuumed, which was followed by filling mercury and increasing the pressure in the container up to the programmed level between 0.5˜500 psi. Once the pressure was forced, mercury penetrated into pores so that the mercury height of the container decreases. This reduction was measured as a (mathematical) function of pressure to calculate the volume of mercury intruded into pores.

As a result, it was found that the final form of the ECM scaffold had the average pore diameter and porosity of 113±26 μm (77˜147 μm range) and 90±10.4% (78˜106%) (n=6), respectively.

Since high porosity of the scaffold provides a larger surface area for cell adhesion, this is a very important characteristic (O′Brien, F. J. et al., Biomaterials, 26:433, 2005). From the results, it was ascertained that the ECM scaffold according to the present invention is useful for tissue engineering applications because the scaffold possesses over 90% porosity in average.

### Example 8

### SEM (Scanning Electron Microscope) Analysis

In order to analyze the microstructure of ECM scaffold sections, a specimen was placed on an aluminum stub with a double-stick carbon tape and transferred to sputtering system (Sanyu Denshi, Tokyo, Japan), then each specimen was coated with 60% gold and 40% palladium with the thickness of 20 nm for 2 minutes.

Also, in order to observe chondrocytes seeded to the scaffold in Example 6, the chondrocytes were fixed with 2.5% glutaldehyde in 0.1M PBS buffer at 4° C. for 2 hours. A control group was fixed later within 12-hour post-seeding so as to compare a morphological change with time. The fixed cells were dehydrated with a series of alcohol concentrations (70˜100%) and washed with PBS twice, followed by cutting each specimen in half with a razor blade. After coating the cross sections for 2 minutes with a sputter coater which is an ion coater, SEM (JSM-6400Fs; JEOL, Tokyo, Japan) analysis was carried out.

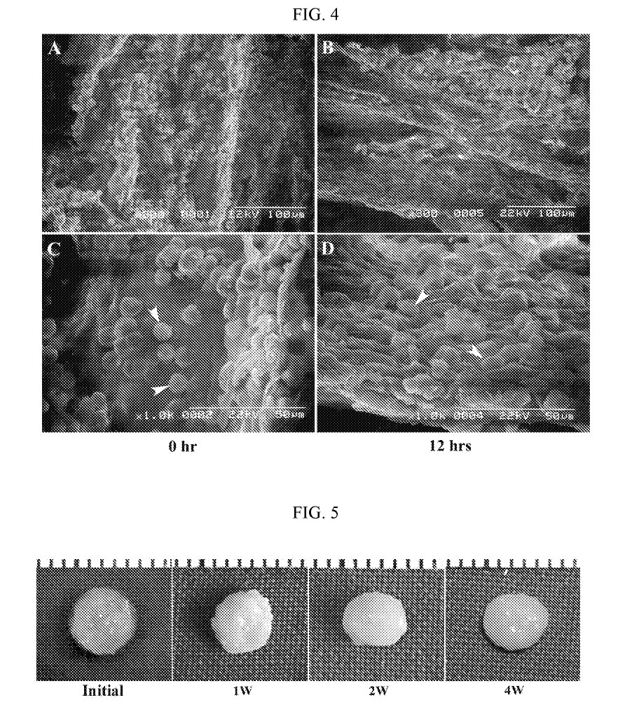

FIG. 4 is SEM images of observing chondrocytes post-seeded to an ECM scaffold at 0 hour (A, C) and 12 hours (B, D) (original magnification, 200× and 1000×, respectively).

It was observed that chondrocytes were attached to the surface of the scaffold at both 0 and 12 hours. At the initial seeding (0 hour), the cell morphology was round as shown as the white arrow in FIG. 4C, but at 12 hours, it turned to be elliptical as shown in FIG. 4D. These results revealed that 12-hour post-seeded chondrocytes are more stably attached to the surface as a flat form.

### Example 9

### Histological Analysis

The neocartilage tissue cultured using the ECM scaffold according to the present invention was fixed with 4% formalin for at least 24 hours in vitro, then embedded in paraffin and sectioned into 4 μm thickness. The cross sections were stained with Safranin O and Alcian blue to detect the sulfated proteoglycan which was accumulated.

FIG. 5 is images of the neocartilage formed based on the ECM scaffold cultured in vitro. Although the chondrocyte-seeded ECM scaffolds were cultured in vitro for 1, 2 and 4 weeks (W), the actual size of neocartilage tissue was not significantly reduced during the whole cultivation time (FIG. 5). As a result of examination with naked eyes, the maturity of the tissue was advanced with the passage of time, and the smooth and glossy surface was observed at 4-week cultivation.

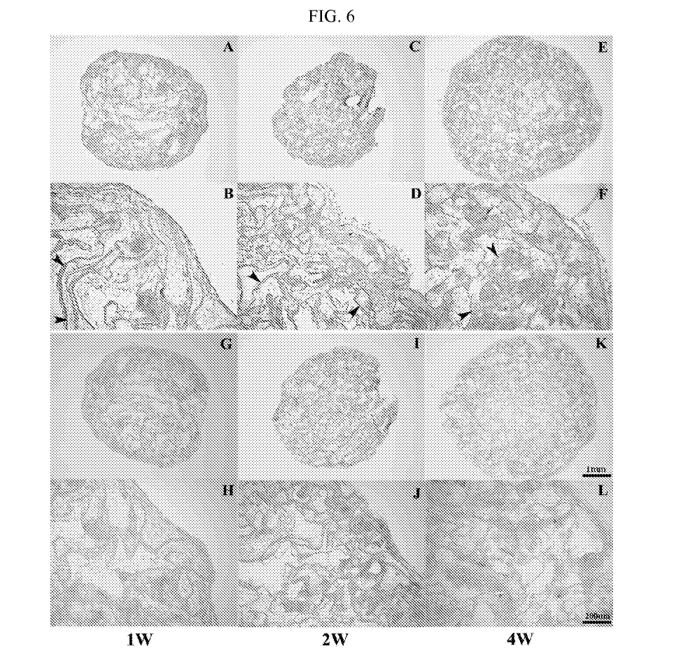

FIG. 6 is images obtained by 20× and 200× magnification to investigate histological features of cartilage tissue fabricated by tissue-engineering through culturing for 1, 2 and 4 weeks. A-F and G-L shows Safranin 0 and Alcian Blue staining, respectively. As shown as the black arrow, the thickness of the ECM scaffold wall became gradually thin with time passage, it is regarded that this phenomenon might be mainly caused by biodegradation of the scaffold (FIGS. 6B, D and F). From the results, it was confirmed that the ECM of cartilage tissue is well-formed and accumulated on the ECM scaffold during the cultivation.

### Example 10

### Immunohistochemical Analysis

For immunohistochemical analysis of type II collagen, the sections prepared in Example 9 were washed with PBS buffer and treated with 3% H₂O₂ for 5 minutes. They were then reacted with 0.15% Triton X-100 to increase tissue permeability.

Once the prepared specimens were blocked with 1% bovine serum albumin (BSA) to suppress nonspecific bindings, the sections were incubated for 1 hour with mouse anti-rabbit collagen type II monoclonal antibody (Chemicon, Temecula, Calif., USA) at 1:200 dilution and then incubated for another 1 hour with 1:200 diluted biotinylated secondary antibody (DAKO LSAB System, Carpinteria, Calif., USA). After being washed with PBS, the section slides were incubated with a peroxidase-conjugated streptavidin solution (DAKO LSAB System) for 30 minutes at ambient temperature. The incubated slides were counterstained with Mayer's hematoxylin (Sigma, St. Louis, Mo., USA) and the slides were mounted with a mount solution for microscopic observation (Nikon E600, Tokyo, Japan).

In order to observe harmonious interaction between collagens of the ECM scaffold and antibodies used in the present invention, immunostaining was performed for only a cell-derived ECM scaffold, like the negative control group untreated with primary antibody and the positive control group treated with both the primary and secondary antibodies.

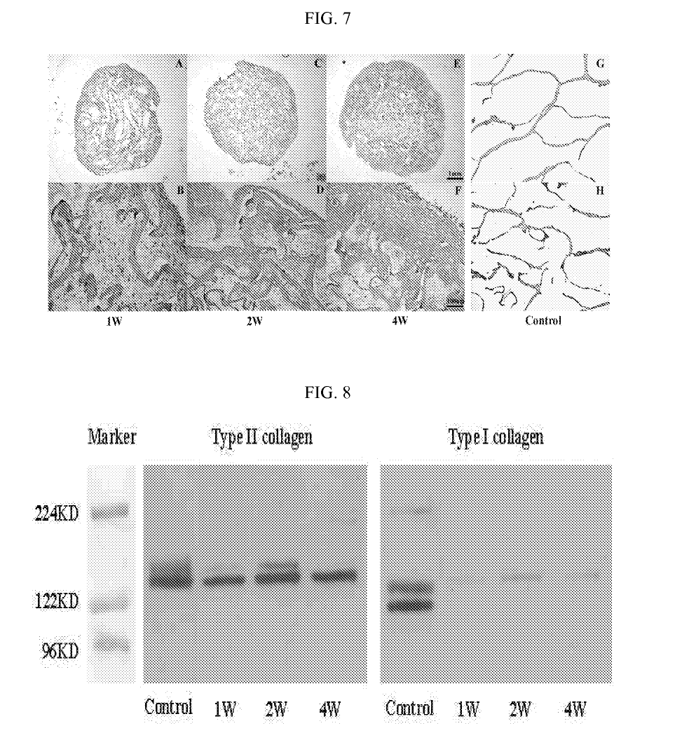

FIG. 7 is images of immunohistochemical analysis of neocartilage tissue cultured for 1, 2 and 4 weeks (observed by a microscope using 20× and 200× magnification). Herein, G is an image of the negative control untreated with the primary antibody and H is an image of the positive control treated with both the primary and secondary antibodies (original magnification, 200×). Additionally, significant difference was not found between the negative control untreated with the primary antibody (G) and the positive control group treated with both the primary and secondary antibodies (H), so that it is affirmed that there is an interaction between proteins extracted from the cell-derived ECM scaffold and antibodies used in the present invention.

### Example 11

### Western Blot Analysis

During the cultivation of the chondrocyte-seeded ECM scaffolds, the formation of type II collagen in the neocartilage tissues was tested with western blot analysis to examine phenotypic stability of neocartilage tissues. Total proteins were extracted from the tissues with a lysis buffer of 40 mM Tris-HCl (pH 8.0) containing 120 mM NaCl, 0.5% Nonidet p-40 (NP-40), 2 μg/ml aprotinin, 2 μg/ml pestetin, 2 μg/ml leupetin, and 100 μg/ml phenylmethylsulfonyl fluoride (PMSF). An equal amount of the proteins quantified by bicinchoninic acid (BCA) method (Shihabi, Z. K. and Dyer R. D., Ann. Clin. Lab. Sci., 18(3):235, 1988) was loaded and separated by 8% sodium dodecyl sulfate-polyacrylamide gel electrophoresis. The separated proteins were then transferred to a nitrocellulose membrane (Millipore, Bedford, Mass., USA). The blotting membrane was incubated first with a mouse anti-rabbit type II collagen monoclonal antibody (Chemicon, Temecular, Calif., USA) diluted at 1:1000 ratio and then rinsed three times with Tris-buffered saline (TBS) containing 0.5% Tween 20, followed by incubating the membrane with a secondary antibody, peroxidase-labeled sheep antimouse IgG (Lockland, Gilbertsville, Pa., USA). The incubated membrane was visualized with an ECL kit (Amersham, N.J., USA).

The immunoblotting analysis was performed by extracting total protein from the ECM scaffolds so as to evaluate interaction of type II collagen monoclonal antibodies. After separating total proteins from SDS-PAGE, chondrocytic phenotype of neocartilage tissues was detected using western blot analysis with type I or II collagen monoclonal antibodies (FIG. 8).

As shown in FIG. 8, the expression of type II collagen was remarkably detected at every experimental group, whereas type I collagen was slightly expressed. However, since neocartilage tissue-derived total proteins were a mixture of newly synthesized proteins with the preexisting proteins, the interaction of the mouse anti-rabbit type II collagen monoclonal antibodies was consistent with the result.

The result exhibited that type II collagen was newly synthesized but could not be completely detected because it was mainly produced by the seeded chondrocytes in vitro cultivation. Therefore, it was found through the western blot analysis that chondrocytes (P1) whose phenotype was maintained in the ECM scaffold can preserve its phenotypic stability at post-translational level.

In the above examples, statistical analysis of the experimental data was performed with one-way analysis of variance for multiple comparisons and Student's t-test (two-tail) for pairwise comparison. The statistical significance was assigned as *P<0.05.

The examples verified that not only the ECM scaffold according to the present invention can stably maintain chondrocytic phenotype through in vitro culture for 4 weeks so that this can have an positive effect on chondrocyte metabolism, but also the ECM scaffold according to the present invention has features of specific structural constructs formed by cartilage-specific ECMs and chondrocytes themselves so that this scaffold is useful as a new scaffold in cartilage tissue engineering.

### INDUSTRIAL APPLICABILITY

As described in detail above, the present invention has an effect to provide an ideal 3D environment where chondrocytes can grow and develop into a high quality cartilage tissues, consequently to provide a method for fabricating a scaffold composed of chondrocytes and their self-produced ECM, and an ECM scaffold fabricated by the same method. A cell-derived ECM scaffold according to the invention is porous, as well as its size is not shrunk during the cultivation after cell seeding so that this scaffold is useful for cartilage transplantation to treat cartilage damages or defects.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. The method of claim 15, wherein the method comprising the steps of:
(a) isolating chondrocytes from animal cartilage and then culturing them;
(b) obtaining a chondrocyte/ECM membrane from the cultured chondrocytes;
(c) obtaining a pellet-type scaffold-free construct by culturing the obtained chondrocyte/ECM membrane; and
(d) obtaining an ECM scaffold by freeze-drying the obtained pellet-type construct.

2. The method of claim 15, wherein the method comprising the steps of:
(a) isolating chondrocytes from animal cartilage and then culturing them;
(b) obtaining a chondrocyte/ECM membrane from the cultured chondrocytes; and
(c) obtaining an ECM scaffold by folding the obtained chondrocyte/ECM membrane or by overlapping several membranes.

3. The method of claim 15, wherein the animal is a pig.

4. The method of claim 15 further comprising adding a growth factor additionally in step (a), the culture step.

5. The method for fabricating a cell-derived ECM scaffold according to claim 4, wherein the growth factor is selected from the group consisting of IGF (insulin-like growth factor), FGF (fibroblast growth factor), TGF (transforming growth factor), BMP (bone morphogenetic protein), NGF (nerve growth factor) and TNF-α (tumor necrosis factor-alpha).

6. The method of claim 15, wherein culture broth is treated with ultrasonic waves or physical pressure is applied to the culture broth in the culture step.

7. The method for fabricating a cell-derived ECM scaffold according to claim 1, wherein the step (c) is performed by fractionating chondrocyte/ECM membrane to collect, and culturing them.

8. The method for fabricating a cell-derived ECM scaffold according to claim 1, wherein the step (d) is performed by repeating 3˜5 times a cycle of freezing and thawing the pellet-type construct at −15˜−25° C. and freeze-drying it.

9. The method for fabricating a cell-derived ECM scaffold according to claim 1, further comprising obtaining a disk-shaped ECM scaffold by processing the obtained ECM scaffold obtained from step (d).

10. A cell-derived porous ECM scaffold fabricated by the method of claim 15, which has pores with a diameter of 10˜1000 μm.

11. A method for fabricating an ECM scaffold similar to natural cartilage or having an excellent mechanical intensity comprising, adding a cartilage component to the ECM scaffold of claim 10 and mixing them.

12. The method for fabricating an ECM scaffold according to claim 11, wherein the cartilage component is collagen or proteoglycan.

13. A method for fabricating an ECM composite scaffold comprising, attaching a biodegradable polymer to the ECM scaffold of claim 10.

14. The method for fabricating an ECM composite scaffold according to the claim 13, wherein the biodegradable polymer is selected from the group consisting of collagen, PLGA (poly-lactic-co-glycolic acid), PLA (polylactate) and PHA (polyhydroxyalkanoate).

15. A method for fabricating a cell-derived ECM scaffold comprising:
(a) isolating chondrocytes from animal cartilage and then culturing them;
(b) obtaining a chondrocyte/ECM membrane from the cultured chondrocytes; and
(c) obtaining an ECM scaffold by obtaining a pellet-type scaffold-free construct by culturing the obtained chondrocyte/ECM membrane, and freeze-drying the obtained pellet-type construct or obtaining an ECM scaffold by folding the obtained chondrocyte/ECM membrane or by overlapping several membranes.
